(19) United States
(12) Patent Application Publication
(10) Pub. No.: US 2010/0317583 A1
(43) Pub. Date: Dec. 16, 2010

(54) TREATMENT OF MELANOMA WITH ALPHA THYMOSIN PEPTIDES IN COMBINATION WITH AN ANTINEOPLASTIC HEAT SHOCK APOPTOSIS ACTIVATOR (HSAA)

(75) Inventors: Israel Rios, Menlo Park, CA (US); Cynthia W. Tuthill, Menlo Park, CA (US)

Correspondence Address: COOLEY LLP;ATTN: Patent Group, Suite 1100, 777 - 6th Street, NW, WASHINGTON, DC, 20001, US

(73) Assignee: SciClone Pharmaceuticals, Inc., Foster City, CA (US)

(21) Appl. No.: 12/747,115

(22) PCT Filed: Dec. 11, 2008
(86) PCT No.: PCT/US08/86427

§ 371 Date: Aug. 20, 2010

Related U.S. Application Data
(60) Provisional application No. 61/013,808, filed on Dec. 14, 2007.

(51) Int. Cl. A61K 38/22; A61P 35/00

(52) U.S. Cl. 514/12.9

(57) ABSTRACT

Melanoma or a metastasis thereof is treated in a human patient in a combination therapy which includes administering a melanoma-treating combination to a human melanoma patient during a treatment regimen, the combination including an alpha thymosin peptide and an antineoplastic heat shock apoptosis activator (HSAA) such as STA-4783 (and optionally an antineoplastic cytotoxic chemotherapeutic agent such as paclitaxel), and/or optionally one or more additional anti-melanoma agents.

### CROSS-REFERENCE TO RELATED APPLICATION(S)

This application claims the benefit of U.S. Provisional Application No. 60/013,808, filed Dec. 14, 2007, the disclosure of which is incorporated herein in its entirety by reference.

### FIELD OF THE INVENTION

The present invention relates to the field of melanoma treatment.

### BACKGROUND OF THE INVENTION

Skin cancer is the most common form of cancer in the United States. In 2007, The American Cancer Society estimates that approximately 8,110 deaths will occur from melanoma and another 59,940 cases of melanoma are expected to be diagnosed in this country.

Melanoma is a malignant tumor of melanocytes which are found predominantly in skin but also in bowel and the eye (uveal melanoma). It is one of the rarer types of skin cancer but causes the majority of skin cancer related deaths.

The treatment includes surgical removal of the tumor; adjuvant treatment; chemo- and immunotherapy, or radiation therapy. Of particular danger are metastases of the primary melanoma tumor.

There remains a need in the art for improved treatments of melanoma.

### SUMMARY OF THE INVENTION

In accordance with the present invention, a method of treating melanoma or a metastasis thereof in a human patient in a combination therapy which comprises administering a melanoma-treating combination to a human melanoma patient during a treatment regimen, the combination comprising an alpha thymosin peptide and an antineoplastic heat shock apoptosis activator (HSAA).

### DESCRIPTION OF THE PREFERRED EMBODIMENTS

The present invention is directed to a method of treating melanoma or metastases thereof in human patients. The method involves administering a melanoma-treating effective combination to human melanoma patients, the combination comprising an alpha thymosin peptide and an antineoplastic heat shock apoptosis activator (HSAA).

In certain embodiments, the combination further includes one or more additional agents to combat or treat melanoma.

Alpha thymosin peptides comprise thymosin alpha 1 (TA1) peptides including naturally occurring TA1 as well as synthetic TA1 and recombinant TA1 having the amino acid sequence of naturally occurring TA1, amino acid sequences substantially similar thereto, or an abbreviated sequence form thereof, and their biologically active analogs having substituted, deleted, elongated, replaced, or otherwise modified sequences which possess bioactivity substantially similar to that of TA1, e.g., a TA1 derived peptide having sufficient amino acid homology with TA1 such that it functions in substantially the same way with substantially the same activity as TA1. Suitable dosages of the alpha thymosin peptide can be within the range of about 0.001-10 mg/kg/day.

The terms “thymosin alpha 1” and “TA1” refer to peptides having the amino acid sequence disclosed in U.S. Pat. No. 4,079,137, the disclosure of which is incorporated herein by reference.

Thymosin alpha 1 (TA1), initially isolated from Thymosin Fraction 5 (TF5), has been sequenced and chemically synthesized. TA1 is a 28 amino acid peptide with a molecular weight of 3108.

Effective amounts of an alpha thymosin peptide are amounts which may be dosage units within ranges corresponding to about 0.1-20 mg of TA1, about 1-10 mg of TA1, about 2-10 mg of TA1, about 2-7 mg of TA1, or about 3-6.5 mg of TA1. Dosage units may comprise about 1.6, 3.2 or 6.4 mg of TA1, or about 3.2 or 6.4 mg of TA1. A dosage unit may be administered once per day, or a plurality of times per day.

Melanoma has various stages, which may include Stage 0, I, II, III and IV, as well as their respective subdivisions. In certain embodiments, the melanoma being treated is malignant metastatic melanoma. In certain embodiments, the melanoma being treated is stage I, stage II, stage III or stage IV. In other embodiments, the melanoma being treated is stage M1a, M1b or M1c melanoma.

The alpha thymosin peptide is administered in a treatment regimen which includes administration to the patient of an antineoplastic heat shock apoptosis activator (HSAA). These include, without limitation, STA-4783 (elesclomol). Elesclomol kills cancer cells by elevating oxidative stress levels beyond a breaking point, triggering programmed cell death. Elesclomol has been shown to rapidly cause a dramatic increase in oxidative stress—the level of reactive oxygen species (ROS)—inside cancer cells.

The method of the present invention comprises administering the alpha thymosin peptide along with administering an antineoplastic heat shock apoptosis activator (HSAA), during a course of the treatment regimen. The HSAA may be administered continuously (i.e., daily), multiple times per day, every other day, etc., and may be administered concurrently with the alpha thymosin peptide or separately therefrom during the treatment regimen, e.g., on the same day(s) as the alpha thymosin peptide or on different days during the course of the treatment regimen. In certain embodiments, the HSAA is administered in dosage ranges of, e.g., about 0.01-1000 mg/kg/day of administration, about 0.1-500 mg/kg/day, or about 1-200 mg/kg/day. Daily dosages may be, e.g., 25 mg/kg, 100 mg/kg, etc.

In certain embodiments, the alpha thymosin peptide is administered in a treatment regimen which includes administration to the patient of an HSAA, the treatment regimen further comprising administration of an antineoplastic cytotoxic chemotherapeutic (CC) agent, such as, without limitation, paclitaxel. Paclitaxel's cytotoxic and anti-tumor properties derive from is ability to promote apoptosis (programmed cell death) by inducing the assembly of microtubules from tubulin dimers and preventing microtubules from depolymerization. The stabilized microtubules inhibit normal dynamic reorganization of the microtubule network that is essential for vital interphase and mitotic functions. In addition paclitaxel induces abnormal arrays or “bundles” of microtubules throughout the cell cycle and multiple asters of microtubules during mitosis. The cytotoxic chemotherapeutic (CC) may be administered to patient in dosage ranges within about 1-500 mg per treatment, or in a dosage range of about 70-280 mg per treatment.

In certain embodiments, the treatment regimen comprises a plurality of days, with the alpha thymosin peptide comprising thymosin alpha 1 (TA1), and the TA1 being administered to the patient during at least a portion of the treatment regimen at a dosage within a range of about 0.5-10 mg/day. In certain embodiments, the TA1 dosage is within a range of about 1.5-7 mg/day, or within a range of about 1.6-6.4 mg/day. In certain embodiments, the TA1 dosage is within ranges of about 1.7-10 mg/day, 1.7-7 mg/day, or about 3-7 mg/day. Exemplary dosages include 1.6, 3.2 and 6.4 mg/day.

In certain embodiments, the treatment regimen comprises administering the alpha thymosin peptide for a period of about 1-10 days, followed by about 1-5 days of non-administration of the alpha thymosin peptide. The alpha thymosin peptide may be administered daily for about 3-5 days, followed by about 2-4 days of non-administration of the alpha thymosin peptide. Alternatively, the alpha thymosin peptide is administered daily for about 4 days, followed by about 3 days of non-administration of the alpha thymosin peptide.

As noted above, in certain embodiments, the combination further includes one or more additional agents to combat or treat melanoma. Such additional agents may be antineoplastic agents such as alkylating antineoplastic agents (AlkAA), which include, without limitation, dacarbazine (DTIC). Additional agent(s) of the combination, such as alkylating antineoplastic agents (AlkAA), may be administered to patient within dosage ranges of, e.g., about 700-1300 mg/m²/day, about 800-1200 mg/m²/day, and/or at about 1000 mg/m²/day.

The various components of the combination may be administered concurrently with, or separately from, other components in a treatment regimen.

According to one embodiment, the invention comprises use of an alpha thymosin peptide and an antineoplastic heat shock apoptosis activator (HSAA) (and optionally an antineoplastic cytotoxic chemotherapeutic agent such as paclitaxel) in manufacture of a melanoma-treating effective pharmaceutical combination or medicament for use in a treatment regimen for treating melanoma or a metastasis thereof in a human melanoma patient.

According to one embodiment, said medicament is for use in a treatment regimen which substantially excludes any immune-stimulating cytokine to said patient during said treatment regimen in an amount significant for treatment of melanoma or a metastasis thereof.

According to one embodiment, the human melanoma patient does not have a substantially elevated LDH blood level, e.g., the LDH blood level is below 475 IU/L.

According to one embodiment, the LDH blood level is between 100-335 IU/L.

One embodiment is the manufacture of a pharmaceutical combination including said alpha thymosin peptide, said combination further comprising an antineoplastic heat shock apoptosis activator (HSAA) (and optionally an antineoplastic cytotoxic chemotherapeutic agent such as paclitaxel) for use during a course of the treatment regimen, which alpha thymosin peptide and antineoplastic heat shock apoptosis activator (HSAA) (and optionally an antineoplastic cytotoxic chemotherapeutic agent such as paclitaxel), and/or optionally one or more additional anti-melanoma agents may be administered separately or together.

According to one embodiment, said HSAA is STA-4783 (elesclomol).

According to one embodiment, the antineoplastic cytotoxic chemotherapeutic agent is paclitaxel.

According to one embodiment, said medicament is for use in a treatment regimen which comprises a plurality of days, said alpha thymosin peptide comprises thymosin alpha 1 (TA1), and said TA1 is for use in administration to said patient during at least a portion of said treatment regimen at a dosage within a range of 0.5-10 mg/day.

According to one embodiment, said TA1 dosage is within a range of 1.5-7 mg/day.

According to one embodiment, said TA1 dosage is 3.2 mg/day.

According to one embodiment, said TA1 dosage is 6.4 mg/day.

According to one embodiment, said alpha thymosin peptide is TA1 and said medicament is for use in a treatment regimen which comprises administration of TA1 daily for a period of about 1-10 days, followed by about 1-5 days of non-administration of said TA1.

According to one embodiment, said TA1 is for use in administration daily for about 3-5 days, followed by about 2-4 days of non-administration of said TA1.

According to one embodiment, said TA1 is for use in administration daily for about 4 days, followed by about 3 days non-administration of said TA1.

The invention also relates to use of an alpha thymosin peptide and an antineoplastic heat shock apoptosis activator (HSAA) (and optionally an antineoplastic cytotoxic chemotherapeutic agent such as paclitaxel) in manufacture of a pharmaceutical combination for administration to a melanoma patient, wherein the alpha thymosin peptide and the antineoplastic heat shock apoptosis activator (HSAA) (and optionally the antineoplastic cytotoxic chemotherapeutic agent such as paclitaxel) may be administered separately or together, as well as to a kit comprising the alpha thymosin peptide, the antineoplastic heat shock apoptosis activator (HSAA) (and optionally the antineoplastic cytotoxic chemotherapeutic agent such as paclitaxel), and/or optionally one or more additional anti-melanoma agents, and further optionally instructions for use in treatment of melanoma.

### Example 1

### An Anti-Tumor Efficacy Study of Combined Treatment of Thymosin Alpha-1, Paclitaxel, and Elesclomol in Mice Bearing Subcutaneous Melanoma

Abbreviations

BW Body Weight

CO₂ Carbon Dioxide

Combo Combination

Ele Elesclomol

G Gram

IR Inhibition Rate

i.v. Intravenous

Kg Kilogram

L Length

Mg Milligram

mL Milliliter

Pac Paclitaxel

PBS Phosphate Buffered Saline

s.c. Subcutaneous

SD Standard Deviation

TA-1 Thymosin Alpha-1

TV Tumor Volume

TW Tumor Weight

BLBF Bridge Laboratories Beijing Facility

W Width

### SUMMARY

In this study, the anti-tumor effect of the combined treatment of thymosin alpha-1 (TA-1), Paclitaxel (Pac) and Elesclomol (Ele) was evaluated in C57BL/6 mice bearing B16 melanoma cells. The toxic effects of the dosing regimens were also monitored. A total of 60 mice were implanted subcutaneously with murine B16 cells, followed by treatment with TA-1 or Pac/Ele alone or in combination for 14 consecutive days. TA-1 was given daily via s.c., while Pac and Ele were co-i.v. administered on Day 1, 7 and 13. In total, 6 groups were used: Group 1: vehicle; Group 2: TA-1 6 mg/kg; Group 3: Pac 10 mg/kg+Ele 10 mg/kg; Group 4: Pac 30 mg/kg+Ele 30 mg/kg; Group 5: TA-1 6 mg/kg+Pac 10 mg/kg+Ele 10 mg/kg; and Group 6: TA-1 6 mg/kg+Pac 30 mg/kg+Ele 30 mg/kg. Tumor volume and body weight were measured every three days, and tumor weights were measured on Day 17 at the end of the study.

Tumor measurement data showed that the mean tumor volumes of all treatment were statistically significantly smaller than that of Group 1 on Days 6, 9, 12 and 15. On Day 17, the mean tumor weights of all treatment groups were lower than Group 1. The PI_{tw}, values of Group 2, Group 3, Group 4, Group 5, and Group 6 were 55.67%, 62.6%, 55.42%, 73.61%, and 54.19%, respectively, indicating effectiveness of all treatment regimens. When TA-1 was used in combination with low-dose Pac/Ele, an enhanced, although statistically insignificant, tumor inhibition effect was observed.

Throughout the course of the study, there were seven animal deaths noticed in Groups 3, 5 and 6, the groups receiving chemotherapeutic treatment. Most of the deaths were observed in the early treatment course when tumors were not palpable. Moreover, the mean body weight in Group 6 (the high dose tri-drug combination treatment group) was significantly decreased. These observations suggested a toxic effect of high dose combination treatment. When used alone, TA-1 did not cause any loss of body weights throughout the course of the study, indicating that TA-1 is not toxic. When combined with low dose Pac/Ele treatment, TA-1 appeared to attenuate the mild loss of body weight caused by the Pac/Ele treatment.

In summary, the tumor model used in this study was valid as tumor growth was inhibited by the positive control drug Pac/Ele. Daily administration of test article TA-1 at 6 mg/kg was effective against the tumor growth. Pac/Ele treatment or with addition of TA-1 were also effective. Higher dose Pac/Ele did not yield an improved tumor inhibition over the low-dose treatment. When TA-1 was combined with low-dose Pac/Ele treatment, an enhanced tumor inhibition and attenuated loss of body weight were observed, indicating beneficial effects.

### INTRODUCTION

Thymosin Alpha-1 (TA-1) is an immunomodulator possessing a potential anti-tumor activity. Paclitaxel (Pac) is a conventional chemotherapeutic drug for various types of cancer. Elesclomol (Ele) is an investigational drug reported to enhance cellular oxidative stress leading to death of cancerous cells. In clinical trials, Ele, in combination with Pac, has demonstrated anti-cancer efficacy in the patients with metastatic melanoma. This study was undertaken to evaluate the efficacy of TA-1 in combination with Pac and Ele towards the B16 melanoma subcutaneously implanted in C57BL/6 mice.

Materials and Methods

Test and Control Articles

PBS was used as the negative control article, and the combination of chemotherapeutic drugs Pac and Ele as the positive control. Pac (Lot# LX-P-070416) was purchased from Knowshine (Shanghai) Pharmachemicals Inc., while Ele (Lot# E08010-34) was supplied by SciClone. Pac and Ele were co-dissolved in the mixture of Cremophor EL/ethanol (50:50; Sigma Cremophor EL, 95% ethyl alcohol) to make two intermediate stock solutions corresponding to concentrations of 24 mg/mL and 8 mg/mL, respectively. The intermediate stock solutions were further diluted with 3 volumes of PBS to make the dosing solutions at 6 mg/mL and 2 mg/mL, respectively (Table 1). Fresh preparation was made for each dosing. TA-1 (SciClone, Lot # PPL-Tα10502) was dissolved in PBS to achieve the proper dose concentration as indicated in Table 1. TA-1 solution was stored at 2-8° C. for a week at most. So in this study two fresh preparations for TA-1 were made.

TABLE 1
Dose Formulation

| | | Dose | |
|---|---|---|---|
| | Dose level | Volume | Concentration |
| Treatment | (mg/kg) | (mL/kg) | (mg/mL) |
| TA-1 | 6 | 5 | 1.2 |
| Pac | 30 | 5 | 6.0 |
| | 10 | 5 | 2.0 |
| Ele* | 30 | 5 | 6.0 |
| | 10 | 5 | 2.0 |

*Ele is to be prepared in combination with Pac.

Test System and Animal Husbandry

Murine B16 Melanoma Cells

Murine B16 melanoma cells were thawed from the stock of Cell Culture Center, Institute of Basic Medical Sciences, Peking Union Medical College and Chinese Academy of Medical Sciences (PUMC & CAMS, Beijing, P. R. China). The tumor cells were adapted in C57BL/6 mice before use in the experiment (Refer to Section 4.3.1 for details on cell adaptation).

Test System

Thirty male and thirty female healthy, naive, C57BL/6 mice were received from the Institute of Laboratory Animal Science, CAMS, Beijing, P. R. China. The animals were six weeks old and weighed between 18 and 22 grams at the start of the study.

Animal Husbandry

Animals were group-housed in autoclaved shoe box cages with autoclaved wood chips as the bedding materials. The temperature of the animal room was maintained at 22 to 25° C., and the relative humidity was maintained at 40 to 60%. A 12-hour light/12-hour dark cycle was maintained except when interrupted by study-related events. Animals were fed ad libitum with sterile water and Beijing KeAoXieLi Rodent Diet (certified). All animals were acclimated for 3 days before tumor inoculation.

Experimental Procedures

Tumor Cell Adaptation

Using aseptic tissue culture procedures, one vial of B16 melanoma cells was removed from the liquid nitrogen stock, and placed into a 37° C. water bath. Gentle swirling was conducted until the content of the vial was thawed. Once thawed, the cells were immediately centrifuged with a TD5A-WS centrifuge at 1000 rpm, 20-25° C., 5 min. After centrifugation, the cells were suspended in 0.1 to 0.5 mL normal saline (NS) and subcutaneously injected into 10 mice (0.1 mL/mouse, about 1×10⁶ cells). After 7-10 days, when the tumor diameter was approximately 1 cm, the animals were euthanized with CO₂ overdose and the tumors excised. The procedure was repeated with 20 mice to generate a sufficient number of B16 melanoma cells with adequate transplantability.

Tumor Cell Inoculation

On the day of tumor implantation, approximately 1×10⁶ cells in 0.1 mL were subcutaneously injected on the right axillary area of each mouse. The day of tumor implantation was defined as Day 0.

Study Design and Treatment Regimen

On Day 1, the animals were randomly assigned into different weight-matched groups, and dosing was started using the regimen according to Table 2. Briefly, TA-1 was administered once daily via subcutaneous (s.c.) injection for 14 consecutive days at a site different from that of tumor cell implantation, while Pac and Ele was administered on Days 1, 7, and 13 via an intravenous (i.v.) injection.

TABLE 2
Treatment Regimen and Study Design

| Group | | | Number | Dosing | Necropsy |
|---|---|---|---|---|---|
| Number | Group Name | Treatment | of Animals | Days | Day |
| 1 | Vehicle Control | PBS | 10 | Days 1-14 | Day 17 |
| 2 | TA-1 | TA-1, 6 mg/kg, s.c., daily | 10 | | |
| 3 | Low Dose | Pac, 10 mg/kg, and Ele, 10 mg/kg, | 10 | | |
| | Pac/Ele | co-i.v., on Days 1, 7, and 13 | | | |
| 4 | High Dose | PAC, 30 mg/kg, and Ele, 30 mg/kg, | 10 | | |
| | Pac/Ele | co-i.v., on Days 1, 7, and 13 | | | |
| 5 | Tri-drug | TA-1, 6 mg/kg, s.c., daily + Pac, 10 mg/kg, | 10 | | |
| | Combo 1 | and Ele, 10 mg/kg, co-i.v., on | | | |
| | | Days 1, 7, and 13 | | | |
| 6 | Tri-drug | TA-1, 6 mg/kg, s.c., daily + Pac, 30 mg/kg, | 10 | | |
| | Combo 2 | and Ele, 30 mg/kg, co-i.v., on | | | |
| | | Days 1, 7, and 13 | | | |

Evaluation of Anti-Tumor Effect

From Day 1 to Day 15, mortality and moribundity were checked twice daily, the body weights were recorded once every 3 days, and tumors were measured using a caliper once every 3 days. At the end of the study (Day 17), the animals were euthanized by CO₂ asphyxiation, and the tumors were excised and weighed.

Based on the tumor size, the tumor volume (TV) was calculated with the formula: [TV=(Length×Width×Width)/2]. And the percent inhibition (PI) of TV (PI_{TV}) was calculated according to the equation below:

PI_{TV}(%)=(TV vehicle−TVdrug treated)/TV vehicle×100

The anti-tumor effect of the test article was further evaluated with tumor weight (TW) measured on day of necropsy (Day 17). The PI of TW was calculated using the equation below:

PI_{TW}(%)=100×(TW vehicle−TW drug treated)/TW vehicle

The calculations of PI_{TV} and PI_{TW} were performed using an Excel spreadsheet and reviewed by the Study Director and Study Monitor.

Evaluation of Treatment Toxicity

Toxicity of all treatment regimens was evaluated with the body weights of the study animals along with the drug-induced animal deaths. The inhibition of body weight was calculated using Excel according to the equation below:

PI_{BW}(%)=100×(BW vehicle−BW drug treated)/BW vehicle

Statistical Analysis

Inter-group comparison was performed in terms of tumor volume, tumor weight and body weight, using a student's t test. P values of less than 0.05 were considered to be statistically significant.

Results

Mortality

Throughout the course of study, there were seven animal deaths. One mouse in Group 5 (Tri-Drug Combo 1) died on Days 2. Two mice in Group 3 (Low Dose Pac/Ele), one in Group 5 and one in Group 6 (Tri-Drug Combo 2) died on Day 3. In addition to the early deaths, one mouse in Group 5 died on Day 11, and one mouse in Group 6 died on Day 14. Most of these deaths occurred in the early treatment course. When the early deaths were noticed, there was no measurable tumor, while significantly decreased body weights were observed in those mice, indicating that these early deaths were linked to the toxic effects of the chemotherapeutic treatments.

Tumor Size

Raw measurement data of tumor size are tabulated in Appendixes 1-10. The calculated mean tumor volumes and statistical testing results of each treatment group versus the vehicle group are tabulated in the Tables 3-7.

On Days 3 and 6 only a few mice had palpable tumors, and there was no statistical difference in tumor volume between the vehicle control group (Group 1) and any treatment group. On Day 9, all mice in Group 1 had palpable tumors. In contrast, only four or five mice in each of treatment groups had tumors. On Day 12 and Day 15, all surviving mice in the Groups 1-6 showed palpable tumors, and the mean tumor volume of each treatment group was statistically significantly smaller than that of Group 1 (p<0.05). Among all treatment groups, Group 5, the group received the combination treatment of TA-1 and Pac/Ele, had the lowest mean tumor volume.

Tumor Weight

Raw data of tumor weights measured on Day 17 are tabulated in Appendix 11. The calculated percent inhibition values based on tumor weight (PI_{tw}) and the statistical comparison results between each of the drug treatment groups and the vehicle group are tabulated in Table 8. As shown in Table 8, the mean tumor weight of each treatment group was lower than that of the vehicle group (Group 1). The PI_{tw} value of Group 2, Group 3, Group 4, Group 5, and Group 6 was 55.67%, 62.6%, 55.42%, 73.61%, and 54.19%, respectively. Consistent with tumor volume data on Day 15, Group 5, the group received the combination treatment of TA-1 and Pac/Ele, had the lowest tumor load.

Body Weight

Raw data of body weight measurement are listed in Appendixes 12-17. The results of statistical comparison of each treatment group versus the vehicle group are tabulated in the Tables 9-14.

As shown in the Tables 9-14, except Group 6 on all time points and Group 4 on Day 9, all other groups and Group 4 on other time points do not show statistically significant (p<0.05) inhibition of body weight relative to the vehicle control group. Since Groups 4 and 6 are the groups that received high dose of Pac/Ele, the results indicate that the chemotherapeutic treatment regimen is associated with toxicity. Compared to the vehicle group, the low-dose Pac/Ele treatment resulted in a mild but statistically insignificant loss of body weight. When the low-dose Pac/Ele was combined with TA-1, the loss of body weight was reduced. This result suggests that TA-1 may attenuate minor toxicity associated with the relatively low-dose chemotherapeutic treatment.

Conclusion and Discussion

In conclusion, the tumor model used in this study was valid as tumor growth was inhibited by the positive control drugs Pac/Ele. Daily administration of TA-1 at 6 mg/kg was effective against the tumor growth. Throughout the course of the study, mean tumor volume in animals of Group 2 which received TA-1 treatment was significantly reduced by more than 50% in comparison to that of the vehicle control group. Mean tumor weight, which was measured on Day 17, were reduced by 55.67% in TA-1-treated animals. Low dose Pac/Ele treatment resulted in 62.6% inhibition of tumor growth based on tumor weight measurement, while that of the high dose Pac/Ele was 55.42%. Higher dose did not yield an improved tumor inhibition. When TA-1 was combined with low-dose Pac/Ele treatment, Group 5 demonstrated 73.61% tumor inhibition, which is higher than that of low-dose Pac/Ele treatment alone (62.60%) or TA-1 treatment alone (55.67%). Although the differences do not reach statistical significance due to higher inter-individual variation, the increased tumor inhibition may suggest an additive effect of TA-1 towards the efficacy of the low dose Pac/Ele treatment. When TA-1 was combined with the high-dose Pac/Ele, there was no additive effect. As a matter of fact, the tumor inhibition rate in the combination treatment group (Group 6) and the high-dose Pac/Ele treatment group (Group 4) was 55.42% and 54.19%, respectively.

When used alone, TA-1 did no cause any statistically significant loss of body weights throughout the course of the study, suggesting that TA-1 is not toxic. In contrast, the mean body weights in Group 6 (high dose tri-drug combination treatment group) were statistically significantly reduced, indicating a toxic effect of chemotherapeutic treatment.

TABLE 3
Statistical results of tumor sizes on Day 3

| | | | Number of | Tumor | | |
|---|---|---|---|---|---|---|
| Group | Group | | Surviving | Volume | PI | P |
| Number | Name | Treatment | Animals | (Mean ± SD) | (TV) | Value |
| 1 | Vehicle | PBS | 10 | 0.0006 ± 0.0012 | NA | NA |
| | Control | | | | | |
| 2 | TA-1 | TA-1, 6 mg/kg, s.c., daily | 10 | 0.0009 ± 0.0016 | −50.00% | 0.6490 |
| 3 | Low Dose | Pac, 10 mg/kg, and Ele, 10 mg/kg, | 8 | 0.0002 ± 0.0003 | 68.75% | 0.3642 |
| | Pac/Ele | co-i.v., on Days 1, 7, | | | | |
| | | and 13 | | | | |
| 4 | High Dose | Pac, 30 mg/kg, and Ele, 30 mg/kg, | 10 | 0.0001 ± 0.0002 | 91.67% | 0.1746 |
| | Pac/Ele | co-i.v., on Days 1, 7, | | | | |
| | | and 13 | | | | |
| 5 | Tri-drug | TA-1, 6 mg/kg, s.c., daily + | 8 | 0.0001 ± 0.0002 | 79.17% | 0.2965 |
| | Combo 1 | Pac, 10 mg/kg, and Ele, 10 mg/kg, | | | | |
| | | co-i.v., on Days 1, 7, | | | | |
| | | and 13 | | | | |
| 6 | Tri-drug | TA-1, 6 mg/kg, s.c., daily + | 9 | 0.0001 ± 0.0002 | 90.74% | 0.2031 |
| | Combo 2 | Pac, 30 mg/kg, and Ele, 30 mg/kg, | | | | |
| | | co-i.v., on Days 1, 7, | | | | |
| | | and 13 | | | | |

TABLE 4
Statistical results of tumor sizes on Day 6

| | | | Number of | Tumor | | |
|---|---|---|---|---|---|---|
| Group | Group | | Surviving | Volume | PI | P |
| Number | Name | Treatment | Animals | (Mean ± SD) | (TV) | Value |
| 1 | Vehicle | PBS | 10 | 0.0053 ± 0.0102 | NA | NA |
| | Control | | | | | |
| 2 | TA-1 | TA-1, 6 mg/kg, s.c., daily | 10 | 0.0022 ± 0.0043 | 58.49% | 0.3889 |
| 3 | Low Dose | Pac, 10 mg/kg, and Ele, 10 mg/kg, | 8 | 0.0002 ± 0.0003 | 96.46% | 0.1796 |
| | Pac/Ele | co-i.v., on Days 1, 7, | | | | |
| | | and 13 | | | | |
| 4 | High Dose | Pac, 30 mg/kg, and Ele, 30 mg/kg, | 10 | 0.0004 ± 0.0013 | 92.45% | 0.1504 |
| | Pac/Ele | co-i.v., on Days 1, 7, | | | | |
| | | and 13 | | | | |
| 5 | Tri-drug | TA-1, 6 mg/kg, s.c., daily + | 8 | 0.0011 ± 0.0018 | 78.77% | 0.2739 |
| | Combo 1 | Pac, 10 mg/kg, and Ele, 10 mg/kg, | | | | |
| | | co-i.v., on Days 1, 7, | | | | |
| | | and 13 | | | | |
| 6 | Tri-drug | TA-1, 6 mg/kg, s.c., daily + | 9 | 0.0001 ± 0.0002 | 97.90% | 0.1479 |
| | Combo 2 | Pac, 30 mg/kg, and Ele, 30 mg/kg, | | | | |
| | | co-i.v., on Days 1, 7, | | | | |
| | | and 13 | | | | |

TABLE 5
Statistical results of tumor sizes on Day 9

| | | | Number of | Tumor | | |
|---|---|---|---|---|---|---|
| Group | Group | | Surviving | Volume | PI | P |
| Number | Name | Treatment | Animals | (Mean ± SD) | (TV) | Value |
| 1 | Vehicle | PBS | 10 | 0.0222 ± 0.0352 | NA | NA |
| | Control | | | | | |
| 2 | TA-1 | TA-1, 6 mg/kg, s.c., daily | 10 | 0.0026 ± 0.0043 | 88.26% | 0.0979 |
| 3 | Low Dose | Pac, 10 mg/kg, and Ele, 10 mg/kg, | 8 | 0.0007 ± 0.0014 | 96.90% | 0.1057 |
| | Pac/Ele | co-i.v., on Days 1, 7, | | | | |
| | | and 13 | | | | |
| 4 | High Dose | Pac, 30 mg/kg, and Ele, 30 mg/kg, | 10 | 0.0013 ± 0.0019 | 94.36% | 0.0768 |
| | Pac/Ele | co-i.v., on Days 1, 7, | | | | |
| | | and 13 | | | | |
| 5 | Tri-drug | TA-1, 6 mg/kg, s.c., daily + | 8 | 0.0016 ± 0.0020 | 92.66% | 0.1207 |
| | Combo 1 | Pac, 10 mg/kg, and Ele, 10 mg/kg, | | | | |
| | | co-i.v., on Days 1, 7, | | | | |
| | | and 13 | | | | |
| 6 | Tri-drug | TA-1, 6 mg/kg, s.c., daily + | 9 | 0.0002 ± 0.0003 | 99.00% | 0.0795 |
| | Combo 2 | Pac, 30 mg/kg, and Ele, 30 mg/kg, | | | | |
| | | co-i.v., on Days 1, 7, | | | | |
| | | and 13 | | | | |

TABLE 6
Statistical results of tumor sizes on Day 12

| | | | Number of | Tumor | | |
|---|---|---|---|---|---|---|
| Group | Group | | Surviving | Volume | PI | P |
| Number | Name | Treatment | Animals | (Mean ± SD) | (TV) | Value |
| 1 | Vehicle | PBS | 10 | 0.1049 ± 0.1226 | NA | NA |
| | Control | | | | | |
| 2 | TA-1 | TA-1, 6 mg/kg, s.c., daily | 10 | 0.0039 ± 0.0038 | 96.28% | 0.0180 |
| 3 | Low Dose | Pac, 10 mg/kg, and Ele, 10 mg/kg, | 8 | 0.0054 ± 0.0067 | 94.87% | 0.0367 |
| | Pac/Ele | co-i.v., on Days 1, 7, | | | | |
| | | and 13 | | | | |
| 4 | High Dose | Pac, 30 mg/kg, and Ele, 30 mg/kg, | 10 | 0.0067 ± 0.0096 | 93.61% | 0.0212 |
| | Pac/Ele | co-i.v., on Days 1, 7, | | | | |
| | | and 13 | | | | |
| 5 | Tri-drug | TA-1, 6 mg/kg, s.c., daily + | 7 | 0.0039 ± 0.0046 | 96.32% | 0.0476 |
| | Combo 1 | Pac, 10 mg/kg, and Ele, 10 mg/kg, | | | | |
| | | co-i.v., on Days 1, 7, | | | | |
| | | and 13 | | | | |
| 6 | Tri-drug | TA-1, 6 mg/kg, s.c., daily + | 9 | 0.0057 ± 0.0046 | 94.54% | 0.0271 |
| | Combo 2 | Pac, 30 mg/kg, and Ele, 30 mg/kg, | | | | |
| | | co-i.v., on Days 1, 7, | | | | |
| | | and 13 | | | | |

TABLE 7
Statistical results of tumor sizes on Day 15

| | | | Number of | Tumor | | |
|---|---|---|---|---|---|---|
| Group | Group | | Surviving | Volume | PI | P |
| Number | Name | Treatment | Animals | (Mean ± SD) | (TV) | Value |
| 1 | Vehicle | PBS | 10 | 0.3739 ± 0.4211 | NA | NA |
| | Control | | | | | |
| 2 | TA-1 | TA-1, 6 mg/kg, s.c., daily | 10 | 0.0539 ± 0.0358 | 85.60% | 0.0277 |
| 3 | Low Dose | Pac, 10 mg/kg, and Ele, 10 mg/kg, | 8 | 0.0512 ± 0.0597 | 86.31% | 0.0483 |
| | Pac/Ele | co-i.v., on Days 1, 7, | | | | |
| | | and 13 | | | | |
| 4 | High Dose | Pac, 30 mg/kg, and Ele, 30 mg/kg, | 10 | 0.0475 ± 0.0323 | 87.31% | 0.0250 |
| | Pac/Ele | co-i.v., on Days 1, 7, | | | | |
| | | and 13 | | | | |
| 5 | Tri-drug | TA-1, 6 mg/kg, s.c., daily + | 7 | 0.0258 ± 0.0184 | 93.10% | 0.0470 |
| | Combo 1 | Pac, 10 mg/kg, and Ele, 10 mg/kg, | | | | |
| | | co-i.v., on Days 1, 7, | | | | |
| | | and 13 | | | | |
| 6 | Tri-drug | TA-1, 6 mg/kg, s.c., daily + | 8 | 0.0563 ± 0.0359 | 84.94% | 0.0506 |
| | Combo 2 | Pac, 30 mg/kg, and Ele, 30 mg/kg, | | | | |
| | | co-i.v., on Days 1, 7, | | | | |
| | | and 13 | | | | |

TABLE 8
Statistical results of tumor weights on Day 17

| | | | Number of | Tumor | | |
|---|---|---|---|---|---|---|
| Group | Group | | Surviving | Weight | PI | P |
| Number | Name | Treatment | Animals | (Mean ± SD) | (TW) | Value |
| 1 | Vehicle | PBS | 10 | 1.99 ± 0.73 | NA | NA |
| | Control | | | | | |
| 2 | TA-1 | TA-1, 6 mg/kg, s.c., daily | 10 | 0.88 ± 0.34 | 55.67% | 0.0004 |
| 3 | Low Dose | Pac, 10 mg/kg, and Ele, 10 mg/kg, | 8 | 0.75 ± 0.43 | 62.60% | 0.0006 |
| | Pac/Ele | co-i.v., on Days 1, 7, and 13 | | | | |
| 4 | High Dose | Pac, 30 mg/kg, and Ele, 30 mg/kg, | 10 | 0.89 ± 0.20 | 55.42% | 0.0002 |
| | Pac/Ele | co-i.v., on Days 1, 7, and 13 | | | | |
| 5 | Tri-drug | TA-1, 6 mg/kg, s.c., daily + Pac, | 7 | 0.53 ± 0.18 | 73.61% | 0.0001 |
| | Combo 1 | 10 mg/kg, and Ele, 10 mg/kg, | | | | |
| | | co-i.v., on Days 1, 7, and 13 | | | | |
| 6 | Tri-drug | TA-1, 6 mg/kg, s.c., daily + Pac, | 8 | 0.91 ± 0.32 | 54.19% | 0.0013 |
| | Combo 2 | 30 mg/kg, and Ele, 30 mg/kg, | | | | |
| | | co-i.v., on Days 1, 7, and 13 | | | | |

TABLE 9
Statistical results of body weights on Day 0

| | | | Number of | Body | | |
|---|---|---|---|---|---|---|
| Group | Group | | Surviving | weight | PI | P |
| Number | Name | Treatment | Animals | (Mean ± SD) | (BW) | Value |
| 1 | Vehicle | PBS | 10 | 20.95 ± 0.98 | NA | NA |
| | Control | | | | | |
| 2 | TA-1 | TA-1, 6 mg/kg, s.c., daily | 10 | 20.98 ± 1.06 | −0.14% | 0.9485 |
| 3 | Low Dose | Pac, 10 mg/kg, and Ele, 10 mg/kg, | 10 | 20.75 ± 0.82 | 0.95% | 0.6273 |
| | Pac/Ele | co-i.v., on Days 1, 7, | | | | |
| | | and 13 | | | | |
| 4 | High Dose | Pac, 30 mg/kg, and Ele, 30 mg/kg, | 10 | 20.47 ± 1.06 | 2.29% | 0.3078 |
| | Pac/Ele | co-i.v., on Days 1, 7, | | | | |
| | | and 13 | | | | |
| 5 | Tri-drug | TA-1, 6 mg/kg, s.c., daily + | 10 | 20.76 ± 1.00 | 0.91% | 0.6730 |
| | Combo 1 | Pac, 10 mg/kg, and Ele, 10 mg/kg, | | | | |
| | | co-i.v., on Days 1, 7, | | | | |
| | | and 13 | | | | |
| 6 | Tri-drug | TA-1, 6 mg/kg, s.c., daily + | 10 | 20.77 ± 1.20 | 0.86% | 0.7180 |
| | Combo 2 | Pac, 30 mg/kg, and Ele, 30 mg/kg, | | | | |
| | | co-i.v., on Days 1, 7, | | | | |
| | | and 13 | | | | |

TABLE 10
Statistical results of body weights on Day 3

| | | | Number of | Body | | |
|---|---|---|---|---|---|---|
| Group | Group | | Surviving | weight | PI | P |
| Number | Name | Treatment | Animals | (Mean ± SD) | (BW) | Value |
| 1 | Vehicle | PBS | 10 | 19.67 ± 0.87 | NA | NA |
| | Control | | | | | |
| 2 | TA-1 | TA-1, 6 mg/kg, s.c., daily | 10 | 19.44 ± 1.12 | 1.17% | 0.6153 |
| 3 | Low Dose | Pac, 10 mg/kg, and Ele, 10 mg/kg, | 8 | 18.51 ± 1.46 | 5.88% | 0.0524 |
| | Pac/Ele | co-i.v., on Days 1, 7, and 13 | | | | |
| 4 | High Dose | Pac, 30 mg/kg, and Ele, 30 mg/kg, | 10 | 18.52 ± 1.83 | 5.85% | 0.0892 |
| | Pac/Ele | co-i.v., on Days 1, 7, and 13 | | | | |
| 5 | Tri-drug | TA-1, 6 mg/kg, s.c., daily + Pac, | 8 | 18.91 ± 1.37 | 3.85% | 0.1715 |
| | Combo 1 | 10 mg/kg, and Ele, 10 mg/kg, | | | | |
| | | co-i.v., on Days 1, 7, and 13 | | | | |
| 6 | Tri-drug | TA-1, 6 mg/kg, s.c., daily + Pac, | 9 | 17.81 ± 1.56 | 9.45% | 0.0048 |
| | Combo 2 | 30 mg/kg, and Ele, 30 mg/kg, | | | | |
| | | co-i.v., on Days 1, 7, and 13 | | | | |

TABLE 11
Statistical results of body weights on Day 6

| | | | Number of | Body | | |
|---|---|---|---|---|---|---|
| Group | Group | | Surviving | weight | PI | P |
| Number | Name | Treatment | Animals | (Mean ± SD) | (BW) | Value |
| 1 | Vehicle | PBS | 10 | 21.54 ± 0.89 | NA | NA |
| | Control | | | | | |
| 2 | TA-1 | TA-1, 6 mg/kg, s.c., daily | 10 | 21.63 ± 0.81 | −0.42% | 0.8158 |
| 3 | Low Dose | Pac, 10 mg/kg, and Ele, 10 mg/kg, | 8 | 20.74 ± 1.04 | 3.73% | 0.0971 |
| | Pac/Ele | co-i.v., on Days 1, 7, | | | | |
| | | and 13 | | | | |
| 4 | High Dose | Pac, 30 mg/kg, and Ele, 30 mg/kg, | 10 | 20.74 ± 1.93 | 3.71% | 0.2494 |
| | Pac/Ele | co-i.v., on Days 1, 7, | | | | |
| | | and 13 | | | | |
| 5 | Tri-drug | TA-1, 6 mg/kg, s.c., daily + | 8 | 20.80 ± 2.57 | 3.44% | 0.4064 |
| | Combo 1 | Pac, 10 mg/kg, and Ele, 10 mg/kg, | | | | |
| | | co-i.v., on Days 1, 7, | | | | |
| | | and 13 | | | | |
| 6 | Tri-drug | TA-1, 6 mg/kg, s.c., daily + | 9 | 20.19 ± 1.69 | 6.27% | 0.0412 |
| | Combo 2 | Pac, 30 mg/kg, and Ele, 30 mg/kg, | | | | |
| | | co-i.v., on Days 1, 7, | | | | |
| | | and 13 | | | | |

TABLE 12
Statistical results of body weights on Day 9

| | | | Number of | Body | | |
|---|---|---|---|---|---|---|
| Group | Group | | Surviving | weight | PI | P |
| Number | Name | Treatment | Animals | (Mean ± SD) | (BW) | Value |
| 1 | Vehicle | PBS | 10 | 21.29 ± 0.72 | NA | NA |
| | Control | | | | | |
| 2 | TA-1 | TA-1, 6 mg/kg, s.c., daily | 10 | 21.05 ± 0.91 | 1.13% | 0.5216 |
| 3 | Low Dose | Pac, 10 mg/kg, and Ele, 10 mg/kg, | 8 | 20.40 ± 1.07 | 4.18% | 0.0511 |
| | Pac/Ele | co-i.v., on Days 1, 7, | | | | |
| | | and 13 | | | | |
| 4 | High Dose | Pac, 30 mg/kg, and Ele, 30 mg/kg, | 10 | 20.11 ± 1.44 | 5.54% | 0.0322 |
| | Pac/Ele | co-i.v., on Days 1, 7, | | | | |
| | | and 13 | | | | |
| 5 | Tri-drug | TA-1, 6 mg/kg, s.c., daily + | 8 | 19.86 ± 2.89 | 6.71% | 0.1491 |
| | Combo 1 | Pac, 10 mg/kg, and Ele, 10 mg/kg, | | | | |
| | | co-i.v., on Days 1, 7, | | | | |
| | | and 13 | | | | |
| 6 | Tri-drug | TA-1, 6 mg/kg, s.c., daily + | 9 | 19.76 ± 1.48 | 7.21% | 0.0095 |
| | Combo 2 | Pac, 30 mg/kg, and Ele, 30 mg/kg, | | | | |
| | | co-i.v., on Days 1, 7, | | | | |
| | | and 13 | | | | |

TABLE 13
Statistical results of body weights on Day 12

| | | | Number of | Body | | |
|---|---|---|---|---|---|---|
| Group | Group | | Surviving | weight | PI | P |
| Number | Name | Treatment | Animals | (Mean ± SD) | (BW) | Value |
| 1 | Vehicle | PBS | 10 | 21.71 ± 0.92 | NA | NA |
| | Control | | | | | |
| 2 | TA-1 | TA-1, 6 mg/kg, s.c., daily | 10 | 21.27 ± 0.91 | 2.03% | 0.5285 |
| 3 | Low Dose | Pac, 10 mg/kg, and Ele, 10 mg/kg, | 8 | 20.89 ± 0.58 | 3.79% | 0.1789 |
| | Pac/Ele | co-i.v., on Days 1, 7, | | | | |
| | | and 13 | | | | |
| 4 | High Dose | Pac, 30 mg/kg, and Ele, 30 mg/kg, | 10 | 21.28 ± 1.53 | 1.98% | 0.5520 |
| | Pac/Ele | co-i.v., on Days 1, 7, | | | | |
| | | and 13 | | | | |
| 5 | Tri-drug | TA-1, 6 mg/kg, s.c., daily + | 7 | 21.20 ± 1.09 | 2.35% | 0.5095 |
| | Combo 1 | Pac, 10 mg/kg, and Ele, 10 mg/kg, | | | | |
| | | co-i.v., on Days 1, 7, | | | | |
| | | and 13 | | | | |
| 6 | Tri-drug | TA-1, 6 mg/kg, s.c., daily + | 9 | 20.43 ± 1.51 | 5.88% | 0.0198 |
| | Combo 2 | Pac, 30 mg/kg, and Ele, 30 mg/kg, | | | | |
| | | co-i.v., on Days 1, 7, | | | | |
| | | and 13 | | | | |

TABLE 14
Statistical results of body weights on Day 15

| | | | Number of | Body | | |
|---|---|---|---|---|---|---|
| Group | Group | | Surviving | weight | PI | P |
| Number | Name | Treatment | Animals | (Mean ± SD) | (BW) | Value |
| 1 | Vehicle | PBS | 10 | 22.35 ± 1.00 | NA | NA |
| | Control | | | | | |
| 2 | TA-1 | TA-1, 6 mg/kg, s.c., daily | 10 | 22.07 ± 0.91 | 1.25% | 0.5205 |
| 3 | Low Dose | Pac, 10 mg/kg, and Ele, 10 mg/kg, | 8 | 21.36 ± 1.22 | 4.42% | 0.0778 |
| | Pac/Ele | co-i.v., on Days 1, 7, | | | | |
| | | and 13 | | | | |
| 4 | High Dose | Pac, 30 mg/kg, and Ele, 30 mg/kg, | 10 | 21.80 ± 1.85 | 2.46% | 0.4193 |
| | Pac/Ele | co-i.v., on Days 1, 7, | | | | |
| | | and 13 | | | | |
| 5 | Tri-drug | TA-1, 6 mg/kg, s.c., daily + | 7 | 21.63 ± 1.03 | 3.23% | 0.1696 |
| | Combo 1 | Pac, 10 mg/kg, and Ele, 10 mg/kg, | | | | |
| | | co-i.v., on Days 1, 7, | | | | |
| | | and 13 | | | | |
| 6 | Tri-drug | TA-1, 6 mg/kg, s.c., daily + | 8 | 20.35 ± 1.72 | 8.95% | 0.0070 |
| | Combo 2 | Pac, 30 mg/kg, and Ele, 30 mg/kg, | | | | |
| | | co-i.v., on Days 1, 7, | | | | |
| | | and 13 | | | | |

### APPENDIX

APPENDIX 1
Tumor measurements (cm) on Day 3

| Group | | F1 | | F2 | | F3 | | F4 | | F5 | | M1 | | M2 | | M3 | | M4 | | M5 | |
|---|---|---|---|---|---|---|---|---|---|---|---|---|---|---|---|---|---|---|---|---|---|
| Number | Group Name | L | W | L | W | L | W | L | W | L | W | L | W | L | W | L | W | L | W | L | W |
| 1 | Vehicle Control | — | — | — | — | 0.1 | 0.1 | — | — | — | — | 0.1 | 0.1 | — | — | 0.2 | 0.2 | 0.1 | 0.1 | 0.1 | 0.1 |
| 2 | TA-1 | — | — | — | — | — | — | — | — | 0.1 | 0.1 | 0.2 | 0.2 | 0.1 | 0.1 | 0.2 | 0.2 | — | — | — | — |
| 3 | Low Dose Pac/Ele | — | — | / | / | 0.1 | 0.1 | — | — | / | / | — | — | — | — | 0.1 | 0.1 | 0.1 | 0.1 | — | — |
| 4 | High Dose Pac/Ele | 0.1 | 0.1 | — | — | — | — | — | — | — | — | — | — | — | — | — | — | — | — | — | — |
| 5 | Tri-drug Como 1 | / | / | — | — | — | — | — | — | — | — | 0.1 | 0.1 | — | — | / | / | 0.1 | 0.1 | — | — |
| 6 | Tri-drug Combo 2 | 0.1 | 0.1 | — | — | — | — | — | — | — | — | — | — | — | — | / | / | — | — | — | — |

Note:
The sign “—” indicates that tumor does not reach a measurable size, while the sign “/” indicates a dead animal.

APPENDIX 2
Tumor volumes (cm³) on Day 3

| Group Number | Group Name | F1 | F2 | F3 | F4 | F5 | M1 | M2 | M3 | M4 | M5 |
|---|---|---|---|---|---|---|---|---|---|---|---|
| 1 | Vehicle Control | 0 | 0 | 0.0005 | 0 | 0 | 0.0005 | 0 | 0.004 | 0.0005 | 0.0005 |
| 2 | TA-1 | 0 | 0 | 0 | 0 | 0.0005 | 0.004 | 0.0005 | 0.004 | 0 | 0 |
| 3 | Low Dose Pac/Ele | 0 | / | 0.0005 | 0 | / | 0 | 0 | 0.0005 | 0.0005 | 0 |
| 4 | High Dose Pac/Ele | 0.0005 | 0 | 0 | 0 | 0 | 0 | 0 | 0 | 0 | 0 |
| 5 | Tri-drug Como 1 | / | 0 | 0 | 0 | 0 | 0.0005 | 0 | / | 0.0005 | 0 |
| 6 | Tri-drug Combo 2 | 0.0005 | 0 | 0 | 0 | 0 | 0 | 0 | / | 0 | 0 |

Note:
The sign “—” indicates that tumor does not reach a measurable size.

APPENDIX 3
Tumor measurements (cm) on Day 6

| Group | | F1 | | F2 | | F3 | | F4 | | F5 | | M1 | | M2 | | M3 | | M4 | | M5 | |
|---|---|---|---|---|---|---|---|---|---|---|---|---|---|---|---|---|---|---|---|---|---|
| Number | Group Name | L | W | L | W | L | W | L | W | L | W | L | W | L | W | L | W | L | W | L | W |
| 1 | Vehicle Control | 0.1 | 0.1 | 0.1 | 0.1 | 0.1 | 0.1 | 0.1 | 0.1 | 0.2 | 0.2 | 0.1 | 0.1 | 0.1 | 0.1 | 0.4 | 0.4 | 0.3 | 0.3 | 0.1 | 0.1 |
| 2 | TA-1 | — | — | — | — | — | — | — | — | 0.3 | 0.3 | 0.2 | 0.2 | 0.1 | 0.1 | 0.2 | 0.2 | — | — | — | — |
| 3 | Low Dose Pac/Ele | — | — | / | / | — | — | — | — | / | / | — | — | — | — | 0.1 | 0.1 | 0.1 | 0.1 | 0.1 | 0.1 |
| 4 | High Dose Pac/Ele | 0.2 | 0.2 | — | — | — | — | — | — | — | — | — | — | — | — | — | — | — | — | — | — |
| 5 | Tri-drug Combo 1 | / | / | 0.1 | 0.1 | — | — | — | — | 0.1 | 0.1- | 0.2 | 0.2 | — | — | / | / | 0.2 | 0.2 | — | — |
| 6 | Tri-drug Combo 2 | 0.1 | 0.1 | — | — | — | — | — | — | — | — | 0.1 | 0.1 | — | — | / | / | — | — | — | — |

Note:
The sign “—” indicates that tumor does not reach a measurable size, while the sign “/” indicates a dead animal.

APPENDIX 4
Tumor volumes (cm³) on Day 6

| Group | | | | | | | | | | | |
|---|---|---|---|---|---|---|---|---|---|---|---|
| Number | Group Name | F1 | F2 | F3 | F4 | F5 | M1 | M2 | M3 | M4 | M5 |
| 1 | Vehicle Control | 0.0005 | 0.0005 | 0.0005 | 0.0005 | 0.004 | 0.0005 | 0.0005 | 0.032 | 0.0135 | 0.0005 |
| 2 | TA-1 | 0 | 0 | 0 | 0 | 0.0135 | 0.004 | 0.0005 | 0.004 | 0 | 0 |
| 3 | Low Dose Pac/Ele | 0 | / | 0 | 0 | / | 0 | 0 | 0.0005 | 0.0005 | 0.0005 |
| 4 | High Dose Pac/Ele | 0.004 | 0 | 0 | 0 | 0 | 0 | 0 | 0 | 0 | 0 |
| 5 | Tri-drug Combo 1 | / | 0.0005 | 0 | 0 | 0.0005 | 0.004 | 0 | / | 0.004 | 0 |
| 6 | Tri-drug Combo 2 | 0.0005 | 0 | 0 | 0 | 0 | 0.0005 | 0 | / | 0 | 0 |

Note:
The sign “/” indicates a dead animal.

APPENDIX 5
Tumor measurements (cm) on Day 9

| Group | | F1 | | F2 | | F3 | | F4 | | F5 | | M1 | | M2 | | M3 | | M4 | | M5 | |
|---|---|---|---|---|---|---|---|---|---|---|---|---|---|---|---|---|---|---|---|---|---|
| Number | Group Name | L | W | L | W | L | W | L | W | L | W | L | W | L | W | L | W | L | W | L | W |
| 1 | Vehicle Control | 0.2 | 0.2 | 0.2 | 0.2 | 0.2 | 0.2 | 0.2 | 0.2 | 0.2 | 0.2 | 0.3 | 0.3 | 0.2 | 0.2 | 0.5 | 0.5 | 0.6 | 0.6 | 0.3 | 0.3 |
| 2 | TA-1 | 0.1 | 0.1 | — | — | — | — | — | — | 0.3 | 0.3 | 0.2 | 0.2 | 0.2 | 0.2 | 0.2 | 0.2 | — | — | — | — |
| 3 | Low Dose Pac/Ele | 0.2 | 0.2 | / | / | 0.1 | 0.1 | 0.1 | 0.1 | / | / | 0.1 | 0.1 | — | — | — | — | — | — | — | — |
| 4 | High Dose Pac/Ele | 0.2 | 0.2 | — | — | 0.1 | 0.1 | — | — | — | — | 0.2 | 0.2 | — | — | 0.2 | 0.2 | — | — | — | — |
| 5 | Tri-drug Combo 1 | / | / | 0.1 | 0.1 | — | — | — | — | 0.2 | 0.2 | 0.2 | 0.2 | 0.1 | 0.1 | / | / | 0.2 | 0.2 | — | — |
| 6 | Tri-drug Combo 2 | 0.1 | 0.1 | 0.1 | 0.1 | — | — | — | — | — | — | — | — | 0.1 | 0.1 | / | / | — | — | 0.1 | 0.1 |

Note:
The sign “—” indicates that tumor does not reach a measurable size, while the sign “/” indicates a dead animal.

APPENDIX 6
Tumor volumes (cm³) on Day 9

| Group | | | | | | | | | | | |
|---|---|---|---|---|---|---|---|---|---|---|---|
| Number | Group Name | F1 | F2 | F3 | F4 | F5 | M1 | M2 | M3 | M4 | M5 |
| 1 | Vehicle Control | 0.004 | 0.004 | 0.004 | 0.004 | 0.004 | 0.0135 | 0.004 | 0.0625 | 0.108 | 0.0135 |
| 2 | TA-1 | 0.0005 | 0 | 0 | 0 | 0.0135 | 0.004 | 0.004 | 0.004 | 0 | 0 |
| 3 | Low Dose Pac/Ele | 0.004 | / | 0.0005 | 0.0005 | / | 0.0005 | 0 | 0 | 0 | 0 |
| 4 | High Dose Pac/Ele | 0.004 | 0 | 0.0005 | 0 | 0 | 0.004 | 0 | 0.004 | 0 | 0 |
| 5 | Tri-drug Combo 1 | / | 0.0005 | 0 | 0 | 0.004 | 0.004 | 0.0005 | / | 0.004 | 0 |
| 6 | Tri-drug Combo 2 | 0.0005 | 0.0005 | 0 | 0 | 0 | 0 | 0.0005 | / | 0 | 0.0005 |

Note:
The sign “/” indicates a dead animal.

APPENDIX 7
Tumor measurements (cm) on Day 12

| Group | | F1 | | F2 | | F3 | | F4 | | F5 | | M1 | | M2 | | M3 | | M4 | | M5 | |
|---|---|---|---|---|---|---|---|---|---|---|---|---|---|---|---|---|---|---|---|---|---|
| Number | Group Name | L | W | L | W | L | W | L | W | L | W | L | W | L | W | L | W | L | W | L | W |
| 1 | Vehicle Control | 0.3 | 0.3 | 0.4 | 0.4 | 0.3 | 0.3 | 0.5 | 0.5 | 0.3 | 0.3 | 0.7 | 0.7 | 0.3 | 0.3 | 0.9 | 0.9 | 0.8 | 0.8 | 0.6 | 0.6 |
| 2 | TA-1 | 0.1 | 0.1 | 0.2 | 0.2 | 0.2 | 0.2 | 0.1 | 0.1 | 0.3 | 0.3 | 0.2 | 0.2 | 0.2 | 0.2 | 0.2 | 0.2 | 0.2 | 0.2 | 0.1 | 0.1 |
| 3 | Low Dose Pac/Ele | 0.3 | 0.3 | / | / | 0.1 | 0.1 | 0.3 | 0.3 | / | / | 0.3 | 0.3 | 0.1 | 0.1 | 0.1 | 0.1 | 0.1 | 0.1 | 0.1 | 0.1 |
| 4 | High Dose Pac/Ele | 0.3 | 0.3 | 0.2 | 0.2 | 0.2 | 0.2 | 0.2 | 0.2 | 0.2 | 0.2 | 0.2 | 0.2 | 0.1 | 0.1 | 0.4 | 0.4 | 0.1 | 0.1 | 0.1 | 0.1 |
| 5 | Tri-drug Combo 1 | / | / | 0.2 | 0.2 | / | / | 0.1 | 0.1 | 0.3 | 0.3 | 0.2 | 0.2 | 0.1 | 0.1 | / | / | 0.2 | 0.2 | 0.1 | 0.1 |
| 6 | Tri-drug Combo 2 | 0.2 | 0.2 | 0.3 | 0.3 | 0.1 | 0.1 | 0.2 | 0.2 | 0.2 | 0.2 | 0.2 | 0.2 | 0.3 | 0.3 | / | / | 0.2 | 0.2 | 0.2 | 0.2 |

Note:
The sign “/” indicates a dead animal.

APPENDIX 8
Tumor volumes (cm³) on Day 12

| Group | | | | | | | | | | | |
|---|---|---|---|---|---|---|---|---|---|---|---|
| Number | Group Name | F1 | F2 | F3 | F4 | F5 | M1 | M2 | M3 | M4 | M5 |
| 1 | Vehicle Control | 0.0135 | 0.032 | 0.0135 | 0.0625 | 0.0135 | 0.1715 | 0.0135 | 0.3645 | 0.256 | 0.108 |
| 2 | TA-1 | 0.0005 | 0.004 | 0.004 | 0.0005 | 0.0135 | 0.004 | 0.004 | 0.004 | 0.004 | 0.0005 |
| 3 | Low Dose Pac/Ele | 0.0135 | / | 0.0005 | 0.0135 | / | 0.0135 | 0.0005 | 0.0005 | 0.0005 | 0.0005 |
| 4 | High Dose Pac/Ele | 0.0135 | 0.004 | 0.004 | 0.004 | 0.004 | 0.004 | 0.0005 | 0.032 | 0.0005 | 0.0005 |
| 5 | Tri-drug Combo 1 | / | 0.004 | / | 0.0005 | 0.0135 | 0.004 | 0.0005 | / | 0.004 | 0.0005 |
| 6 | Tri-drug Combo 2 | 0.004 | 0.0135 | 0.0005 | 0.004 | 0.004 | 0.004 | 0.0135 | / | 0.004 | 0.004 |

Note:
The sign “/” indicates a dead animal.

APPENDIX 9
Tumor measurements (cm) on Day 15

| Group | | F1 | | F2 | | F3 | | F4 | | F5 | | M1 | | M2 | | M3 | | M4 | | M5 | |
|---|---|---|---|---|---|---|---|---|---|---|---|---|---|---|---|---|---|---|---|---|---|
| Number | Group Name | L | W | L | W | L | W | L | W | L | W | L | W | L | W | L | W | L | W | L | W |
| 1 | Vehicle Control | 0.6 | 0.6 | 0.7 | 0.7 | 0.5 | 0.5 | 0.6 | 0.6 | 0.5 | 0.5 | 1.1 | 1.1 | 0.6 | 0.6 | 1.3 | 1.3 | 1.3 | 1.3 | 0.8 | 0.8 |
| 2 | TA-1 | 0.3 | 0.3 | 0.3 | 0.3 | 0.5 | 0.5 | 0.3 | 0.3 | 0.6 | 0.6 | 0.5 | 0.5 | 0.4 | 0.4 | 0.6 | 0.6 | 0.5 | 0.5 | 0.5 | 0.5 |
| 3 | Low Dose Pac/Ele | 0.6 | 0.6 | / | / | 0.3 | 0.3 | 0.7 | 0.7 | / | / | 0.5 | 0.5 | 0.3 | 0.3 | 0.3 | 0.3 | 0.3 | 0.3 | 0.3 | 0.3 |
| 4 | High Dose Pac/Ele | 0.5 | 0.5 | 0.3 | 0.3 | 0.5 | 0.5 | 0.5 | 0.5 | 0.5 | 0.5 | 0.5 | 0.5 | 0.3 | 0.3 | 0.6 | 0.6 | 0.3 | 0.3 | 0.3 | 0.3 |
| 5 | Tri-drug Combo 1 | / | / | 0.4 | 0.4 | / | / | 0.3 | 0.3 | 0.5 | 0.5 | 0.3 | 0.3 | 0.3 | 0.3 | / | / | 0.4 | 0.4 | 0.3 | 0.3 |
| 6 | Tri-drug Combo 2 | 0.5 | 0.5 | 0.6 | 0.6 | 0.3 | 0.3 | 0.4 | 0.4 | 0.4 | 0.4 | 0.5 | 0.5 | 0.6 | 0.6 | / | / | / | / | 0.4 | 0.4 |

Note:
The sign “/” indicates a dead animal.

APPENDIX 10
Tumor volumes (cm³) on Day 15

| Group | | | | | | | | | | | |
|---|---|---|---|---|---|---|---|---|---|---|---|
| Number | Group Name | F1 | F2 | F3 | F4 | F5 | M1 | M2 | M3 | M4 | M5 |
| 1 | Vehicle Control | 0.108 | 0.1715 | 0.0625 | 0.108 | 0.0625 | 0.6655 | 0.108 | 1.0985 | 1.0985 | 0.256 |
| 2 | TA-1 | 0.0135 | 0.0135 | 0.0625 | 0.0135 | 0.108 | 0.0625 | 0.032 | 0.108 | 0.0625 | 0.0625 |
| 3 | Low Dose Pac/Ele | 0.108 | / | 0.0135 | 0.1715 | / | 0.0625 | 0.0135 | 0.0135 | 0.0135 | 0.0135 |
| 4 | High Dose Pac/Ele | 0.0625 | 0.0135 | 0.0625 | 0.0625 | 0.0625 | 0.0625 | 0.0135 | 0.108 | 0.0135 | 0.0135 |
| 5 | Tri-drug Combo 1 | / | 0.032 | / | 0.0135 | 0.0625 | 0.0135 | 0.0135 | / | 0.032 | 0.0135 |
| 6 | Tri-drug Combo 2 | 0.0625 | 0.108 | 0.0135 | 0.032 | 0.032 | 0.0625 | 0.108 | / | / | 0.032 |

Note:
The sign “/” indicates a dead animal.

APPENDIX 11
Tumor weights (g) on Day 17

| Group | | | | | | | | | | | |
|---|---|---|---|---|---|---|---|---|---|---|---|
| Number | Group Name | F1 | F2 | F3 | F4 | F5 | M1 | M2 | M3 | M4 | M5 |
| 1 | Vehicle Control | 1.57 | 2.08 | 1.17 | 1.58 | 1.21 | 2.9 | 1.3 | 2.9 | 2.93 | 2.28 |
| 2 | TA-1 | 0.64 | 0.5 | 1.2 | 0.62 | 1.45 | 0.69 | 0.56 | 1.36 | 0.92 | 0.89 |
| 3 | Low Dose Pac/Ele | 1.14 | / | 0.5 | 1.3 | / | 1.3 | 0.3 | 0.41 | 0.36 | 0.65 |
| 4 | High Dose Pac/Ele | 0.94 | 0.64 | 0.94 | 1.3 | 0.9 | 0.95 | 0.76 | 1.04 | 0.77 | 0.64 |
| 5 | Tri-drug Combo 1 | / | 0.65 | / | 0.59 | 0.7 | 0.3 | 0.4 | / | 0.73 | 0.31 |
| 6 | Tri-drug Combo 2 | 0.9 | 1.34 | 0.43 | 0.77 | 0.77 | 0.86 | 1.4 | / | 0.56 | 0.83 |

Note:
The sign “/” indicates a dead animal.

APPENDIX 12
Body weights (g) on Day 0

| Group | | | | | | | | | | | |
|---|---|---|---|---|---|---|---|---|---|---|---|
| Number | Group Name | F1 | F2 | F3 | F4 | F5 | M1 | M2 | M3 | M4 | M5 |
| 1 | Vehicle Control | 20.7 | 20.6 | 19.6 | 20.0 | 21.4 | 21.8 | 21.5 | 22.7 | 19.8 | 21.4 |
| 2 | TA-1 | 21.5 | 20.8 | 19.3 | 20.0 | 19.9 | 21.8 | 21.5 | 22.9 | 20.7 | 21.4 |
| 3 | Low Dose Pac/Ele | 21.5 | 20.4 | 19.4 | 20.3 | 21.2 | 21.5 | 21.0 | 22.0 | 19.9 | 20.3 |
| 4 | High Dose Pac/Ele | 21.0 | 20.0 | 19.6 | 20.1 | 18.9 | 21.8 | 21.0 | 21.9 | 19.2 | 21.2 |
| 5 | Tri-drug Combo 1 | 20.9 | 20.7 | 19.7 | 20.7 | 19.4 | 22.0 | 21.0 | 22.7 | 20.4 | 20.1 |
| 6 | Tri-drug Combo 2 | 20.9 | 20.7 | 18.6 | 21.1 | 21.0 | 21.7 | 20.9 | 23.1 | 19.7 | 20.0 |

APPENDIX 13
Body weights (g) on Day 3

| Group | | | | | | | | | | | |
|---|---|---|---|---|---|---|---|---|---|---|---|
| Number | Group Name | F1 | F2 | F3 | F4 | F5 | M1 | M2 | M3 | M4 | M5 |
| 1 | Vehicle Control | 19.8 | 18.6 | 19.4 | 19.2 | 18.6 | 20.0 | 20.0 | 20.7 | 19.1 | 21.3 |
| 2 | TA-1 | 17.5 | 19.0 | 20.2 | 19.8 | 19.1 | 21.1 | 17.8 | 19.7 | 20.4 | 19.8 |
| 3 | Low Dose Pac/Ele | 20.4 | / | 17.2 | 18.1 | 18.7 | 20.7 | 18.7 | 18.4 | 16.4 | 18.2 |
| 4 | High Dose Pac/Ele | 19.9 | 16.1 | 16.7 | 16.3 | 17.1 | 20.9 | 19.3 | 20.6 | 18.6 | 19.7 |
| 5 | Tri-drug Combo 1 | / | 18.5 | 15.8 | 19.5 | 18.9 | 20.0 | 19.6 | / | 19.0 | 20.0 |
| 6 | Tri-drug Combo 2 | 16.5 | 15.9 | 15.6 | 19.3 | 18.2 | 19.7 | 19.4 | / | 17.2 | 18.5 |

Note:
The sign “/” indicates a dead animal.

APPENDIX 14
Body weights (g) on Day 6

| Group | | | | | | | | | | | |
|---|---|---|---|---|---|---|---|---|---|---|---|
| Number | Group Name | F1 | F2 | F3 | F4 | F5 | M1 | M2 | M3 | M4 | M5 |
| 1 | Vehicle Control | 21.6 | 20.8 | 20.5 | 20.3 | 21.5 | 22.0 | 22.2 | 23.0 | 21.0 | 22.5 |
| 2 | TA-1 | 20.6 | 21.2 | 21.2 | 20.7 | 21.5 | 22.8 | 22.3 | 22.7 | 21.1 | 22.2 |
| 3 | Low Dose Pac/Ele | 21.4 | / | 19.5 | 20.4 | / | 21.0 | 21.6 | 19.0 | 22.0 | 21.0 |
| 4 | High Dose Pac/Ele | 20.5 | 18.4 | 19.2 | 19.0 | 18.2 | 23.5 | 22.3 | 22.6 | 21.6 | 22.1 |
| 5 | Tri-drug Combo 1 | / | 21.3 | 14.8 | 20.8 | 20.3 | 22.6 | 22.9 | / | 21.7 | 22.0 |
| 6 | Tri-drug Combo 2 | 19.9 | 18.2 | 16.6 | 21.6 | 21.2 | 21.1 | 21.0 | / | 20.9 | 21.2 |

Note:
The sign “/” indicates a dead animal.

APPENDIX 15
Body weights (g) on Day 9

| Group | | | | | | | | | | | |
|---|---|---|---|---|---|---|---|---|---|---|---|
| Number | Group Name | F1 | F2 | F3 | F4 | F5 | M1 | M2 | M3 | M4 | M5 |
| 1 | Vehicle Control | 21.5 | 20.6 | 20.7 | 20.2 | 21.4 | 22.3 | 21.4 | 22.4 | 20.8 | 21.6 |
| 2 | TA-1 | 20.4 | 20.7 | 20.4 | 20.2 | 20.0 | 22.5 | 21.2 | 22.5 | 20.9 | 21.7 |
| 3 | Low Dose Pac/Ele | 20.9 | / | 19.2 | 19.6 | / | 21.9 | 21.0 | 21.3 | 18.9 | 20.4 |
| 4 | High Dose Pac/Ele | 19.5 | 18.1 | 19.3 | 19.0 | 18.5 | 22.3 | 20.8 | 21.9 | 20.6 | 21.1 |
| 5 | Tri-drug Combo 1 | / | 21.0 | 13.0 | 19.6 | 20.1 | 22.0 | 21.8 | / | 20.4 | 21.0 |
| 6 | Tri-drug Combo 2 | 19.4 | 18.1 | 16.6 | 20.9 | 20.4 | 20.7 | 20.6 | / | 20.3 | 20.8 |

Note:
The sign “/” indicates a dead animal.

APPENDIX 16
Body weights (g) on Day 12

| Group | | | | | | | | | | | |
|---|---|---|---|---|---|---|---|---|---|---|---|
| Number | Group Name | F1 | F2 | F3 | F4 | F5 | M1 | M2 | M3 | M4 | M5 |
| 1 | Vehicle Control | 22.1 | 20.8 | 21.0 | 20.4 | 21.3 | 23.0 | 22.3 | 22.8 | 21.0 | 22.4 |
| 2 | TA-1 | 21.5 | 21.3 | 20.2 | 20.1 | 20.1 | 22.4 | 21.5 | 22.4 | 21.0 | 22.2 |
| 3 | Low Dose Pac/Ele | 20.6 | / | 20.3 | 20.4 | / | 21.5 | 20.7 | 22.0 | 20.8 | 20.8 |
| 4 | High Dose Pac/Ele | 20.0 | 19.8 | 20.6 | 20.0 | 19.5 | 23.4 | 22.4 | 23.7 | 21.5 | 21.9 |
| 5 | Tri-drug Combo 1 | / | 20.8 | / | 19.2 | 20.7 | 22.2 | 22.4 | / | 21.4 | 21.7 |
| 6 | Tri-drug Combo 2 | 20.1 | 18.6 | 17.3 | 21.4 | 21.0 | 21.5 | 21.0 | / | 21.3 | 21.7 |

Note:
The sign “/” indicates a dead animal.

APPENDIX 17
Body weights (g) on Day 15

| Group | | | | | | | | | | | |
|---|---|---|---|---|---|---|---|---|---|---|---|
| Number | Group Name | F1 | F2 | F3 | F4 | F5 | M1 | M2 | M3 | M4 | M5 |
| 1 | Vehicle Control | 22.5 | 21.3 | 21.4 | 21.4 | 21.7 | 23.7 | 22.6 | 24.1 | 21.8 | 23.0 |
| 2 | TA-1 | 21.7 | 21.5 | 21.2 | 21.2 | 21.1 | 22.5 | 22.5 | 23.4 | 22.0 | 23.6 |
| 3 | Low Dose Pac/Ele | 21.6 | / | 20.8 | 19.9 | / | 22.7 | 19.6 | 23.0 | 22.0 | 21.3 |
| 4 | High Dose Pac/Ele | 21.0 | 19.6 | 20.5 | 20.1 | 19.5 | 24.2 | 23.4 | 23.9 | 23.3 | 22.5 |
| 5 | Tri-drug Combo 1 | / | 21.2 | / | 20.5 | 20.2 | 22.5 | 23.0 | / | 22.0 | 22.0 |
| 6 | Tri-drug Combo 2 | 18.1 | 20.0 | 17.4 | 21.0 | 21.5 | 21.1 | 21.8 | / | / | 21.9 |

Note:
The sign “/” indicates a dead animal.

### Example 2

In this regimen, TA1 is administered to melanoma patients in a treatment regimen at a dosage within a range of 0.5-10 mg/day.

The melanoma patients also are treated with STA-4783 (elesclomol) at a dose level of 25 mg/kg or 100 mg/kg daily.

### Example 3

In this regimen, TA1 is administered to melanoma patients in a treatment regimen at a dosage within a range of 0.5-10 mg/day.

The melanoma patients also are treated with STA-4783 (elesclomol) at a dose level of 25 mg/kg or 100 mg/kg daily.

The melanoma patients additionally are treated with paclitaxel at a dosage within a range of about 70-280 mg per treatment, or about 1-15 mg/kg/day (e.g., about 7.5 mg/kg/day).

1. A method of treating melanoma or a metastasis thereof in a human patient in a combination therapy which comprises administering a melanoma-treating effective combination to a human melanoma patient during a treatment regimen, the combination comprising an alpha thymosin peptide and an antineoplastic heat shock apoptosis activator (HSAA).

2. The method of claim 1 wherein said HSAA comprises STA-4783 (elesclomol).

3. The method of claim 1 wherein said treatment regimen comprises a plurality of days, said alpha thymosin peptide comprises thymosin alpha 1 (TA1), and said TA1 is administered to said patient during at least a portion of said treatment regimen at a dosage within a range of about 0.5-10 mg/day.

4. The method of claim 3 wherein said dosage is within a range of about 1.5-7 mg/day.

5. The method of claim 3 wherein said dosage is within a range of about 3-7 mg/day.

6. The method of claim 3 wherein said dosage is about 3.2 mg/day.

7. The method of claim 3 wherein said dosage is about 6.4 mg/day.

8. The method of claim 1 wherein said alpha thymosin peptide is TA1 and said treatment regimen comprises administration of TA1 daily for a period of about 1-10 days, followed by about 1-5 days of non-administration of said TA1.

9. The method of claim 8 wherein said TA1 is administered daily for about 3-5 days, followed by about 2-4 days of non-administration of said TA1.

10. The method of claim 8 wherein said TA1 is administered daily for about 4 days, followed by about 3 days non-administration of said TA1.

11. The method of claim 1 wherein said HSAA is administered to said patient at a dosage within a range of about 0.01-1000 mg/kg/day.

12. The method of claim 1 wherein said HSAA is administered to said patient at a dosage of about 1-200 mg/kg/day.

13. The method of claim 1, wherein said combination further includes administration of an antineoplastic cytotoxic chemotherapeutic (CC) agent.

14. The method of claim 13 wherein the CC agent comprises paclitaxel.

15. The method of claim 13 wherein the CC agent is administered to said patient at a dosage within a range of about 1-500 mg.

16. The method of claim 13 wherein the CC agent is administered to said patient at a dosage within a range of about 1-15 mg/kg/day.
